(19) United States
(12) Patent Application Publication
(10) Pub. No.: US 2019/0159977 A1
(43) Pub. Date: May 30, 2019

(54) UV BLOCK COSMETIC COMPOSITION IN THE FORM OF OIL-IN-WATER EMULSION OR WATER-IN-OIL EMULSION AND PREPARATION METHOD THEREOF

(71) Applicant: SOULBRAIN CO., LTD., Seongnam-si (KR)

(72) Inventors: SeungHyun LEE, Seongnam-si (KR); Seok Joo KIM, Seongnam-si (KR)

(21) Appl. No.: 16/172,873

(22) Filed: Oct. 29, 2018

(30) Foreign Application Priority Data
Nov. 29, 2017 (KR) 10-2017-0161627

(51) Int. Cl. A61K 8/06; A61K 8/27; A61K 8/29; A61K 8/34; A61Q 17/04

(52) U.S. Cl. CPC A61K 8/062; A61K 8/064; A61K 8/27; A61K 2800/412; A61K 8/345; A61Q 17/04; A61K 2800/413; A61K 8/29

(57) ABSTRACT

The present invention discloses an ultraviolet (UV) block cosmetic composition in the form of a water-in-oil (W/O) emulsion in which a discontinuous water phase is uniformly distributed in a continuous oil phase, or in the form of an oil-in-water (O/W) emulsion in which a discontinuous oil phase is uniformly distributed in a continuous water phase, the UV block cosmetic composition comprising a water phase, an oil phase, and an emulsifier, wherein the water phase includes a water phase medium and cerium oxide particles, and the oil phase includes any one or more selected from a group consisting of an oil phase medium, a hydrophobic UV blocking agent, and an oil UV blocking agent. A UV block cosmetic composition according to the present invention has a superior UVA and UVB blocking effect and a good feeling of use.

### CROSS-REFERENCE TO RELATED APPLICATIONS

This application claims priority under 35 U.S.C. § 119 to Korean Patent Application No. 10-2017-0161627, filed on Nov. 29, 2017, in the Korean Intellectual Property Office, the disclosure of which is incorporated herein by reference in its entirety.

### TECHNICAL FIELD

The following disclosure relates to an ultraviolet (UV) block cosmetic composition in the form of oil-in-water (O/W) emulsion or water-in-oil (W/O) emulsion and a preparation method thereof, and more specifically, to a UV block cosmetic composition in which a water phase containing cerium oxide particles and an oil phase are mixed in a hybrid emulsion form, and a preparation method thereof.

### BACKGROUND

Ultraviolet (UV) blocks have been used to reduce skin damage caused by photons emitted from sunlight and artificial light. Representative kinds of skin damage include sunburn, which is caused by exposure of a skin layer to UVB, and sunlight aging, which is caused by exposure to UVA up to the dermal layer. In the past, ‘sunscreens,’ referred to as UV blocks, were applied to the skin during outdoor activities to reduce sunburn. However, in recent years, cosmetics including UV block have been applied to the skin for the purpose of providing beauty throughout everyday life. UVB is attenuated by passage through glass, but UVA cannot be blocked by a glass window, thereby resulting in sunlight aging due to exposure to ultraviolet rays even in a car or indoors. In other words, as the skin may be exposed to sunlight aging without distinguishing between indoors and outdoors, application of cosmetics containing a UV block at all times may be a better method of protecting the skin. In order to effectively block ultraviolet rays, both the blocking performance of the UV blocking agent itself and the synergistic effects obtained with application in a formulation may be considered, and it is preferable to consider skin trouble and potential harmfulness when used in cosmetics.

UV blocks may be largely divided into inorganic UV blocks and organic UV blocks, and each blocking component has a blending limit determined by the Food and Drug Administration. Considering the blending limit of the UV block, organic UV blocks may have higher potential harmful effects to the human body, such as inducing skin trouble, than inorganic UV blocks. Therefore, research on technology for producing UV block cosmetics implementing an inorganic UV block more effectively than organic UV blocks is emerging as a big challenge.

Most creams are emulsifications which contain a water phase and an oil phase that are mixed. When UV block ingredients are injected into both the water phase and oil phase, the blocking components are uniformly distributed throughout a dispersion medium, and thus it is possible to expect a synergistic UV blocking effect. However, while most organic and inorganic UV blocks can be easily injected into the oil phase, to date, there is little research on the technology of injecting an organic UV block into the water phase. The inorganic UV blocking agents titanium dioxide and zinc oxide have inherently hydrophilic surfaces which are capable of being dispersed in water, but due to their dense powder texture and poor feeling of use they are mostly subjected to hydrophobic surface treatment, and thus it is general to perform oil dispersion. Potassium cetyl phosphate is added thereto as a dispersion stabilizer in order to forcefully induce dispersion in water. However, there are problems in that improvement of the dispersion stability, feeling of use, and UV blocking ability are low, and there is a difference in the synergistic effect obtained when both the water phase and oil phase contain UV block compared to that obtained from the particles with an inherently hydrophilic surface. Thus, there is a need to develop a technology for preparing a UV block by injecting an inorganic UV blocking agent into a water phase without any surface treatment or pretreatment.

Therefore, to solve the aforementioned problems, the present inventors studied the preparation of a UV block by injecting an inorganic UV block into a water phase without any surface treatment or pretreatment, found that when a hybrid emulsion is prepared by dispersing cerium oxide particles without surface modification in the water phase medium and subsequently mixing with an oil phase, a UV block cosmetic composition with a remarkable increase in the UV blocking effect and increased formulation stability can be produced, and thus completed the present invention.

### SUMMARY

An embodiment of the present invention is directed to providing an ultraviolet (UV) block cosmetic composition.

Another embodiment of the present invention is directed to providing a preparation method of a UV block cosmetic composition.

The technical objectives to be achieved by the present invention are not limited to those mentioned above, and other technical aspects that are not mentioned will be clear to those skilled in the art from the following description.

To achieve the above-mentioned objectives, an embodiment of the present invention provides a UV block cosmetic composition in the form of a water-in-oil (W/O) emulsion in which a discontinuous water phase is uniformly distributed in a continuous oil phase, or in the form of an oil-in-water (O/W) emulsion in which a discontinuous oil phase is uniformly distributed in a water phase, the UV block cosmetic composition comprising a water phase, an oil phase, and an emulsifier, wherein the water phase includes a water phase medium and cerium oxide particles, and the oil phase includes any one or more selected from a group consisting of an oil phase medium, a hydrophobic UV blocking agent, and an oil UV blocking agent.

Here, the cerium oxide particles may have a secondary particle size of 30 to 200 nm.

The cerium oxide particles may have one or more forms selected from a group consisting of a cubic form, a hexagonal form, a polygonal form, a spherical form, and an aggregated spherical form.

The content of the cerium oxide particles may be 1 to 20 wt % based on the total weight of the UV block cosmetic composition.

The cerium oxide particles may have a powder purity of 98 to 99.9%.

The cerium oxide particles may have a surface charge zeta potential value of 20 to 50 mV.

The hydrophobic UV blocking agent may be one or more selected from a group consisting of zinc oxide particles and titanium dioxide particles.

The content of the hydrophobic UV blocking agent may be 0 to 30 wt % based on the total weight of the UV block cosmetic composition.

The oil UV blocking agent may be one or more selected from a group consisting of 4-Methylbenzylidene Camphor, Drometrizole, Drometrizole Siloxane, Digalloyl Trioleate, Disodium Phenyl Dibenzimidazole Tetrasulfonate Acid, Diethylamino Hydroxybenzoyl Hexyl Benzoate, Diethylamino Hydroxybenzoyl Hexyl Benzoate, Diethylhexyl Butamido Triazone, Methylene Bis-Benzotriazolyl Tetramethylbutylphenol, Menthyl Anthranilate, Benzophenone-3, Benzophenone-4, Benzophenone-8, Butyl Methoxydibenzoylmethane, Bis-EthylhexyloxyphenolMethoxyphenyl Triazine, Cinoxate, Ethylhexyl Dimethyl PABA, Ethylhexyl Methoxycinnamate, Ethylhexyl Salicylate, Ethylhexyl Triazone, Octocrylene, Isoamyl p-Methoxycinnamate, Terephthalylidene Dicamphor Sulfonic Acid, Phenylbenzimidazole Sulfonic Acid, Polysilicone-15, and homosalate.

The content of the oil UV blocking agent may be 0 to 25 wt % based on the total weight of the UV block cosmetic composition.

The water phase medium may be purified water or an acidic aqueous solution with a pH of 5 to 7.

The content of the water phase medium may be 10 to 50 wt % based on the total weight of the UV block cosmetic composition.

The oil phase medium may be one or more selected from a group consisting of ethylene glycol, diethylene glycol, propylene glycol, dipropylene glycol, butylene glycol, ester oil, mineral oil, and silicone oil.

The content of the oil phase medium may be 10 to 50 wt % based on the total weight of the UV block cosmetic composition.

The emulsifier may be a non-ionic emulsifier.

The content of the emulsifier may be 1 to 5 wt % based on the total weight of the UV block cosmetic composition.

The UV block cosmetic composition may further comprise a cosmetic formulation composition.

Another embodiment of the present invention provides a method for the preparation of a UV block cosmetic composition comprising: dispersing cerium oxide particles in purified water to prepare a supernatant; adding and stirring the supernatant in a water phase medium to prepare a water phase raw material; adding and stirring any one or more selected from a group consisting of a hydrophobic UV blocking agent and an oil UV blocking agent in an oil phase medium to prepare an oil phase raw material; and stirring an outer phase and then injecting an inner phase, wherein the outer phase and the inner phase are each independently a water phase raw material or an oil phase raw material, and the outer and inner phases are different raw materials from each other.

### BRIEF DESCRIPTION OF THE DRAWINGS

FIG. 1 is a schematic diagram showing a water-in-oil (W/O) formulation and an oil-in-water (O/W) formulation of an ultraviolet (UV) block cosmetic composition according to an aspect of the present invention.

FIG. 2 is a graph showing the sun protection factor (SPF) measurement value of Example 1.

FIG. 3 is a graph showing the SPF measurement value of Example 2.

FIG. 4 is a graph showing the SPF measurement value of Comparative Example 1.

FIG. 5 is a graph showing the SPF measurement value of Comparative Example 2.

FIG. 6 is a graph showing the UVA PF indexes according to the cerium oxide particle content ratios of Example 1, Example 2 and Comparative Example 1.

FIG. 7 is a graph showing critical wavelength values according to the cerium oxide particle content ratios of Example 1, Example 2 and Comparative Example 1.

### DETAILED DESCRIPTION OF EMBODIMENTS

Hereinafter, a detailed description of the present invention is provided. It should be understood, however, that the following description is an example only, and the present invention is only limited by the scope of the following claims.

In addition, the terms used in the invention are only used to describe certain embodiments, and they are not intended to limit the scope of the invention. The expression of singular or plural words is only made in context of grammar, and can include both meanings. Unless specifically stated otherwise, expressions such as “including” or “comprising” do not indicate the exclusion of all other components, and additional components may also be included.

An embodiment of the present invention provides an ultraviolet (UV) block cosmetic composition in the form of a water-in-oil (W/O) emulsion in which a discontinuous water phase is uniformly distributed in a continuous oil phase, or in the form of an oil-in-water (O/W) emulsion in which a discontinuous oil phase is uniformly distributed in a continuous water phase, the UV block cosmetic composition comprising a water phase, an oil phase, and an emulsifier, wherein the water phase includes a water phase medium and cerium oxide particles, and the oil phase includes any one or more selected from a group consisting of an oil phase medium, a hydrophobic UV blocking agent, and an oil UV blocking agent.

Hereinafter, the UV block cosmetic composition is described in detail.

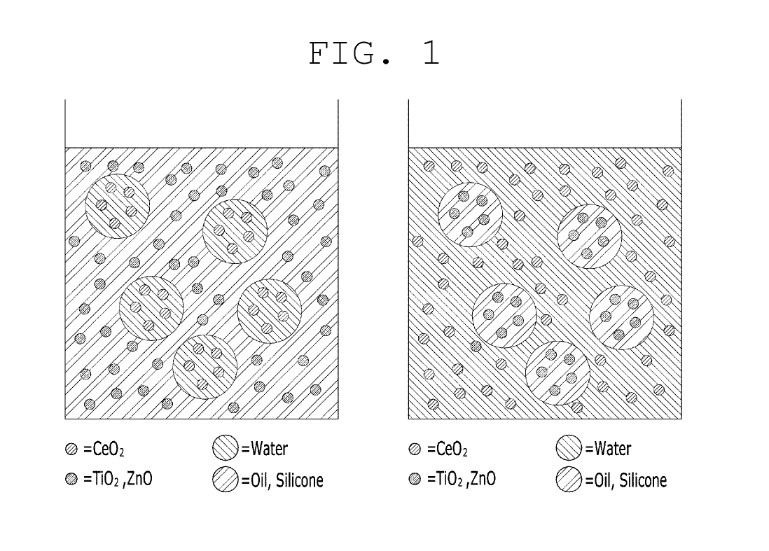

FIG. 1 is a schematic diagram showing a water-in-oil (W/O) formulation and an oil-in-water (O/W) formulation of a UV block cosmetic composition according to an aspect of the present invention.

As shown in FIG. 1, the UV block cosmetic composition according to an aspect of the present invention may have a formulation in the form of a water-in-oil (W/O) emulsion, in which an oil phase distributed as a continuous phase is applied as an outer phase and a water phase which is discontinuously distributed in the outer phase is applied as an inner phase, or a formulation in the form of an oil-in-water (O/W) emulsion, in which a water phase distributed as a continuous phase is applied as an outer phase and an oil phase which is discontinuously distributed in the outer phase is applied as an inner phase.

Here, emulsion refers to a formulation in the form of an emulsified liquid, and means a solution in which two kinds of liquids which do not dissolve each other are dispersed in a colloidal state in another liquid. More specifically, emulsion means a state in which one of the two liquids which do not dissolve each other is dispersed in a small particle state in the other liquid, and for example, the two liquids which do not dissolve each other may indicate water and oil. The emulsion may have the form of an oil-in-water (O/W) emulsion in which the oil phase is dispersed in the water phase, and the form of a water-in-oil (W/O) emulsion which is opposite to the O/W emulsion.

In the UV block cosmetic composition according to an aspect of the present invention, the water phase comprises a water phase medium and cerium oxide particles.

Here, the water phase medium may be purified water or an acidic aqueous solution with a pH of 5 to 7.

The content of the water phase medium may be 1 to 60 wt %, preferably 5 to 55 wt %, and more preferably 10 to 50 wt %, based on the total weight of the UV block cosmetic composition.

The content of the water phase medium may be freely selected within the above-described range depending on the formulation of the UV block cosmetic composition to be prepared. For example, when it is attempted to prepare a water-in-oil (W/O) UV block cosmetic composition in a cream form with a high oil content, the composition may be prepared in the form of an emulsion in which a discontinuous water phase is distributed in a continuous oil phase by decreasing the content of the water phase medium and increasing the content of the oil phase medium. When it is attempted to prepare an oil-in-water (O/W) UV block cosmetic composition in a lotion form with a high water content, the composition may be prepared in the form of an emulsion in which a discontinuous oil phase is distributed in a continuous water phase by increasing the content of the water phase medium and decreasing the content of the oil phase medium.

The water phase medium may serve as a solvent capable of dispersing cerium oxide particles which have a hydrophilic surface so as to allow them to be present in the cosmetic composition formulation, and simultaneously may allow the selection of various kinds of formulations of the UV block cosmetic composition by appropriately adjusting the content ratio thereof.

In addition, the cerium oxide particles employed may be cerium oxide particles obtained by calcination, precipitation, coprecipitation or hydrothermal synthesis using cerium hydroxide, cerium carbonate, cerium nitrate, cerium chloride, ammonium cerium nitrate, a hydrate thereof, or the like. There is no particular limitation to the production method of the cerium oxide particles, and any generally used cerium oxide particles may be used without being limited to the above-described production methods.

The cerium oxide particles may have a secondary particle size of 20 to 250 nm, preferably 25 to 230 nm, and more preferably 30 to 200 nm.

When the secondary particle size of the cerium oxide particles is less than 30 nm, the UV blocking effect may be insufficient due to the excessively small size of the particles and when the secondary particle size thereof exceeds 200 nm, the feeling of use such as powder texture, or the like, of the cosmetic composition may be lowered due to the excessively large particle size. Here, secondary particle size means the average size of the particle formed by agglomerating the cerium oxide particles, each having a primary particle size.

The cerium oxide particles may have a cubic form, a hexagonal form, a polygonal form, a spherical form, and an aggregated spherical form, and may be a mixture of cerium oxide particles having the above-described forms. However, the form and shape of cerium oxide particles are not limited to the above-described kinds.

The content of the cerium oxide particles may be 0.1 to 25 wt %, preferably 1 to 23 wt %, and more preferably 1 to 20 wt %, based on the total weight of the UV block cosmetic composition.

When the content of the cerium oxide particles is less than 1 wt %, the UV blocking effect of the cosmetic composition may be deteriorated due to the excessively low content of the cerium oxide particles. When the content thereof exceeds 20 wt %, the feeling of use such as powder texture, or the like, of the cosmetic composition may be deteriorated due to the excessively high content of the cerium oxide particles.

The cerium oxide particles may have a powder purity of 90 to 99.99%, preferably 95 to 99.9%, and more preferably 98 to 99.9%. When the powder purity of the cerium oxide particles is less than 98%, the skin stability of the cosmetic composition may be lowered due to the presence of by-products other than the cerium oxide particles.

The cerium oxide particles may have a surface charge zeta potential value of 10 to 60 mV, preferably 25 to 45 mV, and more preferably 20 to 50 mV. When the surface charge zeta potential value of the cerium oxide particles is less than 20 mV, the dispersibility may be weakened by ion repulsion, and when the surface charge zeta potential exceeds 50 mV, the cerium oxide particles may exhibit excessively high charge, thus leading to re-agglomeration.

Since the cerium oxide particles are basically an inorganic UV blocking agent having a hydrophilic surface, they are not emulsified in an oil cosmetic composition. However, in the UV block cosmetic composition according to an aspect of the present invention, the cerium oxide particles may be dispersed in the water phase medium without surface modification, thereby resulting in an increase in the dispersibility of the cerium oxide particles, and an oil phase raw material including a hydrophobic inorganic and/or organic UV blocking agent may be mixed thereto, thereby resulting in synergistic effects, facilitating the achievement of a remarkable increase in the UV blocking effect of the UV block cosmetic composition while simultaneously increasing the formulation stability of the composition.

Further, in the UV block cosmetic composition according to an aspect of the present invention, the water phase may further comprise a low-grade alcohol, a polyhydric alcohol, a moisturizer, a pH adjuster, or the like, in addition to the water phase medium and cerium oxide particles.

Examples of the low-grade alcohol may include ethanol, propanol, isopropanol, isobutyl alcohol, t-butyl alcohol, and the like. Examples of the polyhydric alcohol may include dihydric alcohols (e.g., ethylene glycol, propylene glycol, trimethylene glycol, 1,2-butylene glycol, 1,3-butylene glycol, tetramethylene glycol, 2,3-butylene glycol, pentamethylene glycol, 2-butylene-1,4-diol, hexylene glycol, octylene glycol, and the like), trihydric alcohols (e.g., glycerin, trimethylolpropane, and the like), and the like.

Examples of the moisturizer may include polyethylene glycol, propylene glycol, glycerin, 1,3-butylene glycol, xylitol, sorbitol, maltitol, chondroitin sulfate, hyaluronic acid, mucoitin sulfuric acid, caronic acid, atelocollagen, cholesteryl-12-hydroxystearate, sodium lactate, bile salts, dl-pyrrolidone carboxylic acid salt, short-chain soluble collagen, diglycerin (EO)PO adduct, Rosa roxburghii extract, Yarrow extract, Mary Road extract, and the like.

Examples of the pH adjuster may include buffers such as lactic acid-sodium lactate, citric acid-sodium citrate, succinic acid-sodium succinate, and the like, and organic amines such as monoethanolamine, diethanolamine, triethanolamine, tromethamine, and the like.

The combined content of the low-grade alcohol, polyhydric alcohol, moisturizer, and pH adjuster is not particularly limited within the range in which the effects of the present invention are not impaired, and may be about 5 to 25 wt %, preferably about 10 to 20 wt %, and more preferably about 12 to 18 wt %, based on the total weight of the cosmetic composition.

In addition, in the UV block cosmetic composition according to an aspect of the present invention, the oil phase includes an oil phase medium, and also includes a hydrophobic UV blocking agent, an oil UV blocking agent, or a mixture thereof.

Here, the oil phase medium may be ethylene glycol, diethylene glycol, propylene glycol, dipropylene glycol, butylene glycol, ester oil, mineral oil, silicone oil, or the like, or may be a mixture thereof.

Here, the silicone oil may be dimethicone, cetyl dimethicone, cyclopentasiloxane, cyclohexasiloxane, stearyl dimethicone, or the like.

The content of the oil phase medium may be 1 to 60 wt %, preferably 5 to 55 wt %, and more preferably 10 to 50 wt %, based on the total weight of the UV block cosmetic composition.

The content of the oil phase medium may be freely selected from within the above-described range depending on the formulation of the UV block cosmetic composition to be produced. For example, when it is attempted to prepare a water-in-oil (W/O) UV block cosmetic composition in a cream form with a high oil content, the composition may be prepared in the form of an emulsion in which a discontinuous water phase is distributed in a continuous oil phase by increasing the content of the oil phase medium and decreasing the content of the water phase medium. When it is attempted to prepare an oil-in-water (O/W) UV block cosmetic composition in a lotion form with a high water content, the composition may be prepared in the form of an emulsion in which a discontinuous oil phase is distributed in a continuous water phase by decreasing the content of the oil phase medium and increasing the content of the water phase medium.

The oil phase medium may serve as a solvent capable of emulsifying and dispersing the hydrophobic UV blocking agent or the oil UV blocking components in the cosmetic composition formulation, and simultaneously may allow the selection of various kinds of formulations of the UV block cosmetic composition by appropriately adjusting the content ratio thereof.

The hydrophobic UV blocking agent may be zinc oxide particles, titanium dioxide particles, or a mixture thereof. Here, the zinc oxide particles and the titanium dioxide particles may be surface-modified to be hydrophobic. There is no particular limitation on the type of surface modification, and the surface modification is preferably performed to enhance the emulsifiability of the zinc oxide particles and titanium dioxide particles in the oil phase medium within the range in which the effects of the present invention are not impaired.

The particle sizes of the zinc oxide particles and titanium dioxide particles is not particularly limited within the range in which the effects of the present invention are not impaired, and preferably, the zinc oxide and titanium dioxide particles may have secondary particle sizes of 80 nm to 500 nm.

The particle form of the zinc oxide particles and titanium dioxide particles is not particularly limited within the range in which the effects of the present invention are not impaired, and may be preferably a cubic form, a hexagonal form, a polygonal form, a spherical form, and an aggregated spherical form, and may also be a mixture of particles having the above-described forms.

The content of the hydrophobic UV blocking agent may be from 0 to 40 wt %, preferably from 0 to 35 wt %, and more preferably from 0 to 30 wt %, based on the total weight of the UV block cosmetic composition.

The UV block cosmetic composition according to an aspect of the present invention may include only the oil UV blocking agent without including the hydrophobic UV blocking agent. Meanwhile, when the content of the hydrophobic UV blocking agent exceeds 30 wt %, the feeling of use of the cosmetic composition, such as powder texture, or the like, may be lowered due to the excessively high content of the hydrophobic UV blocking agent.

The oil UV blocking agent may be 4-Methylbenzylidene Camphor, Drometrizole Siloxane, Digalloyl Trioleate, Disodium Phenyl Dibenzimidazole Tetrasulfonate Acid, Diethylamino Hydroxybenzoyl Hexyl Benzoate, Diethylamino Hydroxybenzoyl Hexyl Benzoate, Diethylhexyl Butamido Triazone, Methylene Bis-Benzotriazolyl Tetramethylbutylphenol, Menthyl Anthranilate, Benzophenone-3, Benzophenone-4, Benzophenone-8, Butyl Methoxydibenzoylmethane, Bis-EthylhexyloxyphenolMethoxyphenyl Triazine, Cinoxate, Ethylhexyl Dimethyl PABA, Ethylhexyl Methoxycinnamate, Ethylhexyl Salicylate, Ethylhexyl Triazone, Octocrylene, Isoamyl p-Methoxycinnamate, Terephthalylidene Dicamphor Sulfonic Acid, Phenylbenzimidazole Sulfonic Acid, Polysilicone-15 or homosalate), and the like, and a mixture thereof.

Here, the oil UV blocking agent means an organic UV block other than an inorganic UV block, and any organic compound may be applied if it has an ultraviolet blocking ability and is harmless to the human body, in addition to the above-described kinds.

The content of the oil UV blocking agent may be from 0 to 30 wt %, preferably from 0 to 28 wt %, and more preferably from 0 to 25 wt %, based on the total weight of the UV block cosmetic composition, and the content thereof may be adjusted within an appropriate blending range depending on the blending limits of the cosmetics determined for each country.

Further, the UV block cosmetic composition according to an aspect of the present invention includes an emulsifier.

Any emulsifier may be used without limitation to any particular kind if it is an emulsifier capable of forming an emulsion-type formulation by appropriately mixing a water phase raw material and an oil phase raw material within the range in which the effects of the present invention are not impaired, and may be preferably a PEG silicone emulsifier, a non-ionic emulsifier, a cationic emulsifier, an anionic emulsifier, or the like, and more preferably, a non-ionic emulsifier. The content of the emulsifier may be 1 to 10 wt %, preferably 0.1 to 7 wt %, and more preferably 1 to 5 wt %, based on the total weight of the UV block cosmetic composition. When the content of the emulsifier is less than 1 wt %, it may be difficult to form an emulsion due to the excessively low content of the emulsifier. When the content of the emulsifier exceeds 5 wt %, the feeling of use may be inhibited due to the feeling of oiliness caused by the excessively high content of the emulsifier.

In addition, the UV block cosmetic composition according to an aspect of the present invention may further include a cosmetic formulation composition.

Here, the cosmetic formulation composition may be appropriately blended with ingredients that are generally mixed in cosmetic compositions such as oils, waxes, surfactants, thickeners, pigments, cosmetic additives, powders, saccharides, antioxidants, buffers, various extracts, stabilizers, abirritants, preservatives, moisturizers, fragrances, and the like, within the range in which the effects of the present invention are not impaired. Here, when the cosmetic formulation composition is a hydrophilic material, it may be mixed with the water phase, and when the cosmetic formulation composition is a hydrophobic material, it may be mixed with the oil phase.

The oil may be a vegetable oil such as evening primrose oil, rosehip oil, castor oil, olive oil, and the like, animal oils such as mink oil, squalane, and the like, a mineral oil such as liquid paraffin, vaseline, and the like, or a synthetic oil such as silicone oil, isopropyl myristate oil, and the like.

The wax may be a vegetable wax such as carnauba wax, candelilla wax, jojoba oil, and the like, and an animal wax such as beeswax, lanolin, and the like.

The surfactant may be an anionic surfactant, a cationic surfactant, an amphoteric surfactant, a non-ionic surfactant, and the like.

The thickener may be a water-soluble polymer, and so on.

Here, examples of the water-soluble polymer may include plant-based (polysaccharide-based) natural polymers such as guar gum, locust bean gum, quince seed, carrageenan, galactan, gum arabic, tragacanth gum, pectin, mannan, starch, and the like; microbial (polysaccharide-based) natural polymers such as xanthan gum, dextran, succinol glucan, curdlan, hyaluronic acid, and the like; animal-based (protein-based) natural polymers such as gelatin, casein, albumin, collagen, and the like; cellulose-based semisynthetic polymers such as methyl cellulose, ethyl cellulose, hydroxyethyl cellulose, hydroxypropyl cellulose, carboxymethyl cellulose, methylhydroxypropyl cellulose, and the like; starch-based semisynthetic polymers such as soluble starch, carboxymethyl starch, methyl starch, and the like; alginic acid-based semisynthetic polymers such as alginic acid propylene glycol ester, alginate, and the like; other polysaccharide-based derivative semisynthetic polymers; vinyl-based synthetic polymers such as polyvinyl alcohol, polyvinylpyrrolidone, polyvinylmethylether, carboxyvinyl polymer, sodium polyacrylate, and the like; other synthetic polymers such as polyethylene oxide, ethylene oxide, propylene oxide block copolymers, and the like; and inorganic materials such as bentonite, laponite, microdispersed silicon, colloidal alumina, and the like.

The pigment may be a synthetic pigment or a natural pigment, wherein the synthetic pigment may be: water-soluble/oil-soluble pigments such as FD&C Yellow No. 6, FD&C Red No. 4, and the like; inorganic pigments such as iron oxide, ultramarine, and the like; organic pigments such as D&C Red No. 30, D&C Red No. 36, and the like; and lakes such as FD&C Yellow No. 6 Al lake, and the like; and the natural pigment may be: carotenoid-based pigments such as β-carotene, β-apo-8-carotenal, rilopine, capsanthin, bixin, crocin, canthaxanthin, and the like; flavonoid-based pigments such as shisonin, lamanine, ninocyanine, carthamin, safrole yellow, rutin, quercetin, cocoa pigment, and the like; flavin-based pigments such as riboflavin, and the like, quinone-based pigments such as laccaic acid, carminic acid (cochineal), kermesic acid, alizanine, shikonin, alkannin, nikino chrome, and the like; porphyrin-based pigments such as chromophil, hemoglobin, and the like; diketone-based pigments such as curcumin (turmeric), or the like; and betacyanine-based pigments such as betanine, and the like.

The cosmetic additive may be a vitamin, a plant extract, and/or an animal extract, wherein the vitamin may be retinol (vitamin A), tocopherol (vitamin E), ascorbic acid (vitamin X), and the like; the plant extract may be menthol (peppermint), azulene (chamomile), allantoin (wheat), caffeine (coffee), licorice extract, cinnamon extract, green tea extract, lavender extract, lemon extract, and the like; and the animal extract may be placenta (placenta of cattle), royal jelly (bee secretion), snail extract (mucus secretion), and the like.

As the preservative, nanocolloid, Naturotics, Nafree, Diol PD, methylparaben, Bio-trichlorosilyl, anisic acid natural preservative, ketone CG, paraben, phyto PS, phenoxyethanol, GMCY natural preservative, Naturo Np-3, or the like, may be used.

As the abirritant, ethylhexylglycerin, or the like, may be used.

As the moisturizer, 1,2-hexanediol, glycerin, propylene glycol, butylene glycol, polyethylene glycol, polyols such as sorbitol or trehalose, and the like, natural moisturizing factors (NMFs) such as amino acids, urea, lactate, PCA-Na, and the like, and polymeric moisturizers such as hyaluronate, chondroitin sulfate, hydrolyzed collagen, and the like, moist24, moistin 24 or moistin, and the like, may be used.

As described above, in the UV block cosmetic composition according to an aspect of the present invention, cerium oxide particles with a hydrophobic surface may be dispersed in a hydrophilic solvent without surface modification to form a water phase, and the water phase and oil phase may be mixed to form an emulsion composition in the form of water-in-oil (W/O) or oil-in-water (O/W), thereby resulting in the achievement of a high UV blocking ability and excellent feeling of use of the formulation, and thus the UV block cosmetic composition of the present invention may be effectively used in the production of cosmetics for the purpose of UV protection.

Further, another aspect of the present invention provides a preparation method of a UV block cosmetic composition comprising the steps of: dispersing cerium oxide particles in purified water to prepare a supernatant; adding the supernatant in a water phase medium and stirring to prepare a water phase raw material; adding and stirring in any one or more selected from a group consisting of a hydrophobic UV blocking agent and an oil UV blocking agent in an oil phase medium to prepare an oil phase raw material; and stirring an outer phase while injecting an inner phase, wherein the outer phase and the inner phase are each independently a water phase raw material or an oil phase raw material, and the outer phase and the inner phase are different raw materials from each other.

Hereinafter, the preparation method of a UV block cosmetic composition will be described in detail.

First, the step of dispersing cerium oxide particles in purified water to prepare a supernatant is performed in order to add and disperse the cerium oxide particles, which have a hydrophilic surface, in purified water while simultaneously removing by-products contained in the cerium oxide particles. Here, the kind, size, form, content, and the like, of the cerium oxide particles are the same as described above.

Then, the step of adding and stirring the supernatant in a water phase medium to prepare a water phase raw material is performed by adding the supernatant containing cerium oxide dispersed therein to a water phase medium at an injection rate of 10 to 100 ml/min, thereby preparing the water phase raw material. Here, the kind, content, and the like, of the water phase medium are the same as described above.

Next, the step of adding and stirring in any one or more selected from a group consisting of a hydrophobic UV blocking agent and an oil UV blocking agent in an oil phase medium to prepare an oil phase raw material is performed by adding and dispersing a hydrophobic UV blocking agent, an oil UV blocking agent, or a mixture thereof, in an oil phase medium at an injection rate of 10 to 100 ml/min, thereby preparing the oil phase raw material. Here, the kind, content, and the like, of the oil phase medium, the hydrophobic UV block and the oil UV block, are the same as described above.

Finally, the step of stirring an outer phase while injecting an inner phase is performed in order to mix the prepared water phase raw material and the prepared outer phase raw material, wherein when the formulation of the cosmetic composition to be produced is in the form of oil-in-water (O/W), the water phase raw material may be stirred as the outer phase and the oil phase raw material may be injected as the inner phase, and when the formulation of the cosmetic composition to be produced is in the form of water-in-oil (W/O), the oil phase raw material may be stirred as the outer phase and the water phase raw material may be injected as the inner phase.

Hereinafter, Examples of the present invention will be described in detail so as to be easily practiced by those skilled in the art to which the present invention pertains. However, the present invention may be modified in various different ways and is not limited to these Examples provided in the present description.

### EXAMPLE 1

### Preparation 1 of UV Block Cosmetic Composition

A UV block cosmetic composition in the form of oil-in-water (O/W), according to the present invention, was prepared using zinc oxide particles as a hydrophobic UV blocking agent.

First, 60 g of cerium oxide particles with a particle size of 80 nm was added to 50 g of purified water, and the resultant blend was stirred with ultrasonic waves for 24 hours to disperse the cerium oxide particles in purified water, thereby preparing a cerium oxide particle supernatant.

Next, the blend was stirred at 1000 rpm using a Disper Mixer, 9.09 parts by weight of the prepared cerium oxide particle supernatant was added at an injection rate of 2 ml/min, and the resultant blend was stirred for 30 minutes, thereby preparing a water phase raw material.

Then, the blend was stirred at 1000 rpm using a Disper Mixer, 4 parts by weight of zinc oxide particles (ZN-te 30, manufactured by Hanil Chemical Ind., Co., Ltd.) was added at an injection rate of 1 g/min, and the resultant blend was stirred for 30 minutes, thereby preparing an oil phase raw material.

Finally, as the inner phase, the above-described oil phase raw material was slowly added while stirring the above-described water phase raw material, as the outer phase, and then the resultant blend was emulsified at 2500 rpm for 10 minutes using a homomixer, thereby preparing a UV block cosmetic composition.

### EXAMPLE 2

### Preparation 2 of UV Block Cosmetic Composition

A UV block cosmetic composition according to the present invention was prepared in the same manner as in Example 1, except that 18.18 parts by weight of a cerium oxide particle supernatant was added to prepare the water phase raw material.

### COMPARATIVE EXAMPLE 1

### Preparation 3 of UV Block Cosmetic Composition

A UV block cosmetic composition in the form of oil-in-water (O/W) was prepared without using cerium oxide particles.

First, the blend was stirred for 30 minutes at 500 rpm using a Disper Mixer to prepare a water phase raw material.

Then, the blend was stirred at 1000 rpm using a Disper Mixer, 4 parts by weight of zinc oxide particles (ZN-te 30, manufactured by Hanil Chemical Ind., Co., Ltd.) was added at an injection rate of 1 g/min, and the resultant blend was stirred for 30 minutes, thereby preparing an oil phase raw material.

Finally, as the inner phase, the above-described oil phase raw material was slowly added while stirring the above-described water phase raw material, as the outer phase, and then the resultant blend was emulsified at 2500 rpm for 10 minutes using a homomixer, thereby preparing a UV block cosmetic composition.

### COMPARATIVE EXAMPLE 2

### Preparation 4 of UV Block Cosmetic Composition

A UV block cosmetic composition in the form of oil-in-water (O/W) was prepared using titanium oxide particles instead of cerium oxide particles.

First, the blend was stirred at 1000 rpm using a Disper stirrer, 25 parts by weight of a titanium oxide aqueous dispersion (T40AQS, manufactured by SUNJIN Beauty Science Co., LTD.) was added at an injection rate of 2 ml/min, and the blend was then stirred for 30 minutes, thereby preparing a water phase raw material.

Then, the blend was stirred at 1000 rpm using a Disper Mixer, 4 parts by weight of zinc oxide particles (ZN-te 30, manufactured by Hanil Chemical Ind., Co., Ltd.) was added at an injection rate of 1 g/min, and the resultant blend was stirred for 30 minutes, thereby preparing an oil phase raw material.

Finally, as the inner phase, the above-described oil phase raw material was slowly added while stirring the above-described water phase raw material, as the outer phase, and then the resultant blend was emulsified at 2500 rpm for 10 minutes using a homomixer, thereby preparing a UV block cosmetic composition.

The ingredients and content ratios of the UV block cosmetic compositions prepared in Examples 1 and 2 and Comparative Examples 1 and 2 are summarized in Table 1 below.

TABLE 1

| | | | Composition ratio (wt %) | | | |
|---|---|---|---|---|---|---|
| Classification | | Ingredient name | | | Comparative | Comparative |
| Classification | | Ingredient name | Example 1 | Example 2 | Example 1 | Example 2 |
| Water phase | Hydrophilic | Cerium oxide | 9.09 | 18.18 | — | — |
| raw | UV block | particles | | | | |
| material | | 55 wt % aqueous | | | | |
| | | dispersion | | | | |
| | Water | Purified water | 43.06 | 36.97 | 50.15 | 30.15 |
| | phase | | | | | |
| | medium | | | | | |
| | Moisturizer | Glycerin | 5 | 5 | 5 | 5 |
| Oil phase | Hydrophobic | Zinc oxide | 4 | 4 | 4 | 4 |
| raw | UV block | powder | | | | |
| material | | (NZ-te 30) | | | | |
| | | Titanium oxide | — | — | — | 25 |
| | | particles | | | | |
| | | 5 wt % aqueous | | | | |
| | | dispersion | | | | |
| | Oil UV | Ethylhexyl | 7 | 7 | 7 | 7 |
| | block | methoxycinnamate | | | | |
| | | (Parsol MCX) | | | | |
| | | Ethylhexyl | 3 | 3 | 3 | 3 |
| | | salicylate | | | | |
| | | (Parsol EHS) | | | | |
| | Oil | Dipropylene | 10 | 10 | 10 | 10 |
| | phase | glycol | | | | |
| | medium | | | | | |
| | | Cyclopentasiloxane | 2.5 | 2.5 | 3 | 2.5 |
| | | (Sf 1202) | | | | |
| | | Ester oil | 10 | 7 | 12 | 7 |
| | | (Dermofeel bgc) | | | | |
| Emulsifier | | Non-ionic W/O | 3 | 3 | 3 | 3 |
| | | emulsifier | | | | |
| | | (Abil em 90) | | | | |
| Cosmetic | Thickener | Planar mica | 3 | 3 | 2.5 | 3 |
| formulation | | (Bentone gel | | | | |
| composition | | CGF) | | | | |
| | Abirritant | Ethylhexylglycerin | 0.05 | 0.05 | 0.05 | 0.05 |
| | | (Sensiva sc-50) | | | | |
| | Preservative | Phenoxyethanol | 0.3 | 0.3 | 0.3 | 0.3 |

| | | | | |
|---|---|---|---|---|
| Total | 100 | 100 | 100 | 100 |

### EXPERIMENTAL EXAMPLE 1

### SPF Measurement of UV Block Cosmetic Composition

Experiments were performed to measure and compare the SPF values of the UV block cosmetic compositions prepared in Examples 1 and 2 and Comparative Examples 1 and 2, respectively, in order to confirm the UVB blocking ability.

The SPF indexes of the UV block cosmetic compositions prepared in Examples 1 and 2 and Comparative Examples 1 and 2 were measured six times using an Optometrics SPF-290A Analyzer. The average measured SPF index values of the respective compositions are summarized and shown in Table 2 below.

TABLE 2

| Classification | SPF value |
|---|---|
| Example 1 | 27.535 |
| Example 2 | 41.1 |
| Comparative Example 1 | 15.075 |
| Comparative Example 2 | 31.22 |

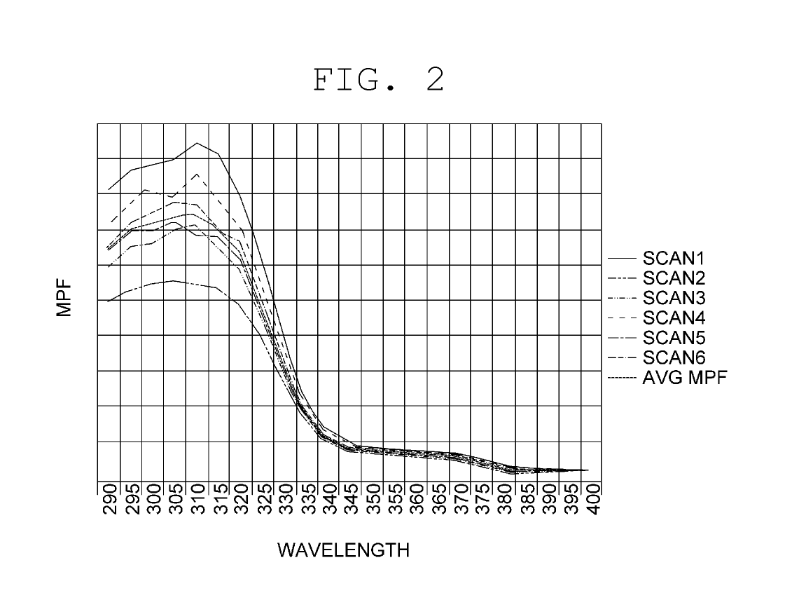

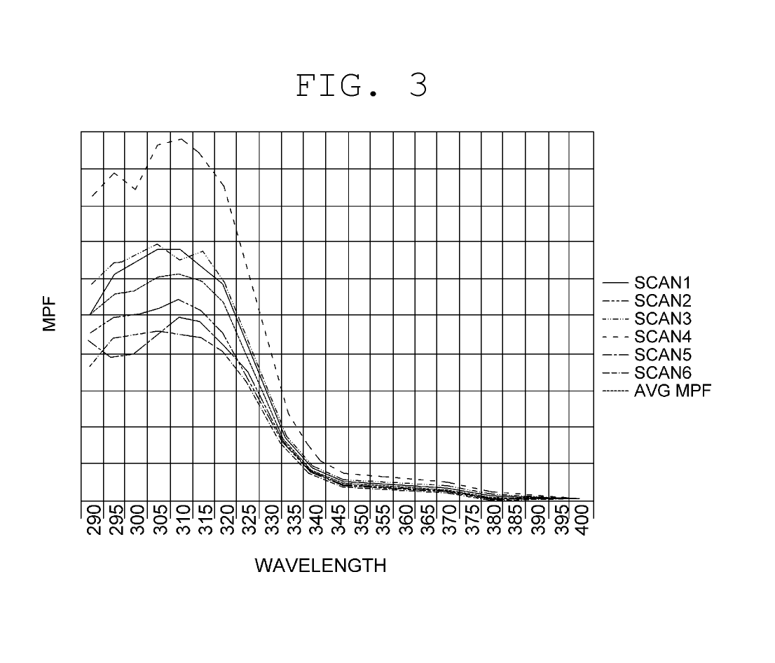

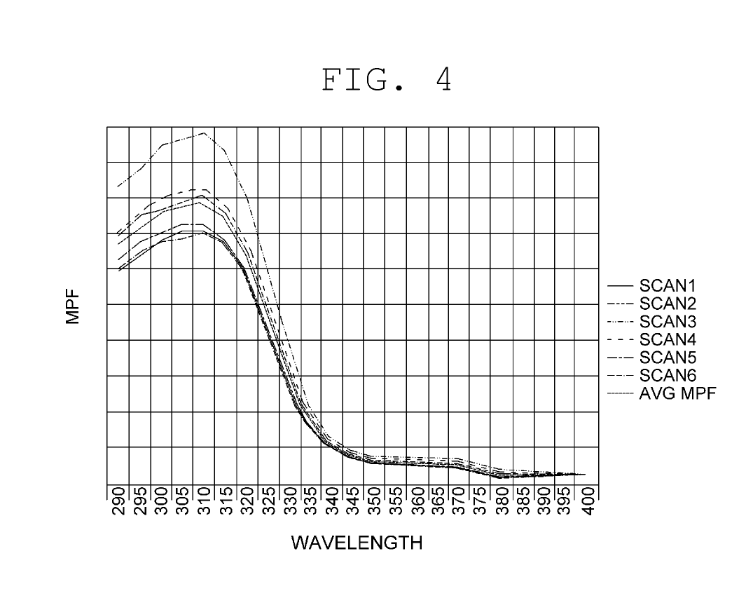

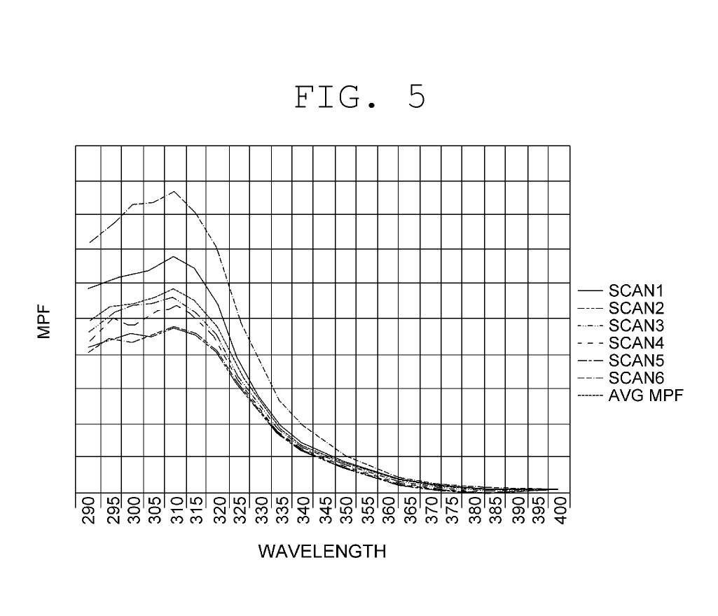

FIG. 2 is a graph showing the sun protection factor (SPF) measurement values of Example 1, FIG. 3 is a graph showing the SPF measurement values of Example 2, FIG. 4 is a graph showing the SPF measurement values of Comparative Example 1, and FIG. 5 is a graph showing the SPF measurement values of Comparative Example 2.

As can be confirmed from Table 2 and FIGS. 2 to 5, the UV block cosmetic composition according to an aspect of the present invention had excellent UV blocking ability.

### EXPERIMENTAL EXAMPLE 2

### UVA PF Measurement of UV Block Cosmetic Composition

Experiments were performed to measure and compare the UVA protection factor (PF) values of the UV block cosmetic compositions prepared in Examples 1 and 2 and Comparative Examples 1 and 2, respectively, in order to confirm the UVA blocking ability.

The UVA PF indexes of the UV block cosmetic compositions prepared in Examples 1 and 2 and Comparative Examples 1 and 2 were measured six times using an Optometrics SPF-290A Analyzer. The measured UVA PF index average values of the respective compositions are summarized and shown in Table 3 below.

TABLE 3

| Classification | UVA PF value |
|---|---|
| Example 1 | 10.375 |
| Example 2 | 16.33 |
| Comparative Example 1 | 5.565 |
| Comparative Example 2 | 9.112 |

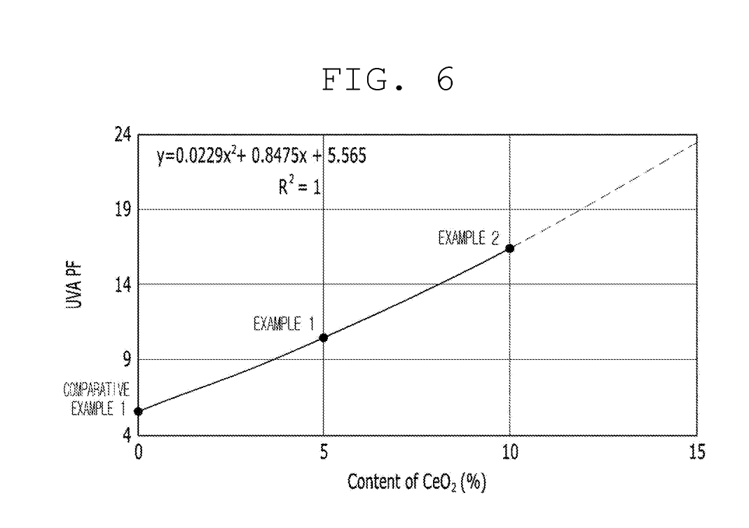

FIG. 6 is a graph showing the UVA PF indexes according to the cerium oxide particle content ratios of Example 1, Example 2 and Comparative Example 1.

As can be confirmed from Table 3 and FIG. 6, the UV block cosmetic composition according to an aspect of the present invention had excellent UVA blocking ability.

### EXPERIMENTAL EXAMPLE 3

### Measurement of Critical Wavelength of UV Block Cosmetic Composition

Experiments were performed to measure and compare the critical wavelength values of the UV block cosmetic compositions prepared in Examples 1 and 2 and Comparative Examples 1 and 2, respectively, in order to confirm what range of wavelengths the UV block cosmetic compositions are capable of blocking from reaching the skin.

Specifically, the UV block cosmetic compositions prepared in Examples 1 and 2 and Comparative Examples 1 and 2 were uniformly applied to a test subject, and the critical wavelength values were measured by irradiating light with the UV Protection Measurement Equipment SPF-290A. The measured critical wavelength values of the respective compositions are summarized in Table 4 below.

TABLE 4

| | Critical wavelength |
|---|---|
| Classification | value |
| Example 1 | 376.9 |
| Example 2 | 377.7 |
| Comparative Example 1 | 367 |
| Comparative Example 2 | 371 |

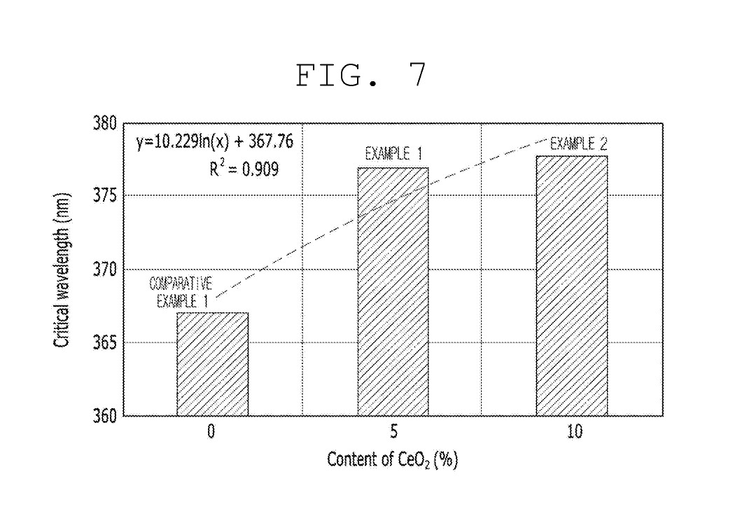

FIG. 7 is a graph showing the critical wavelength values of Example 1, Example 2 and Comparative Example 1, according to the cerium oxide particle content ratios.

As can be confirmed from Table 4 and FIG. 7, the UV block cosmetic composition according to an aspect of the present invention had a high critical wavelength blocking ability of 376 nm or more, thereby resulting in the achievement of excellent blocking ability in the long wavelength UV region which may cause skin aging.

Thus, the UV block cosmetic composition according to an aspect of the present invention may be effectively used as a UV block cosmetic composition for preventing skin aging.

### EXPERIMENTAL EXAMPLE 4

### Measurement of Feeling of Use of UV Block Cosmetic Composition

Experiments were performed to measure and compare the feelings of use of the UV block cosmetic compositions prepared in Examples 1 and 2 and Comparative Examples 1 and 2, respectively, in order to confirm the feeling of use when the compositions were actually applied to the skin.

The experiments were performed on 10 test subjects with neutral skin. The UV block cosmetic compositions prepared in Examples 1 and 2 and Comparative Examples 1 and 2 were each applied in an amount of 0.5 ml to the back of the hand, and then the degree of stiffness in the powder texture of the composition when rubbed with the index finger 20 times was determined to evaluate the general feeling of use on a scale of 5 points. The average values of the evaluated scores are summarized in Table 5 below.

TABLE 5

| | General feeling of use |
|---|---|
| Classification | (Up to 5 points) |
| Example 1 | 4.77 |
| Example 2 | 4.75 |
| Comparative Example 1 | 4.8 |
| Comparative Example 2 | 3.21 |

As can be confirmed from Table 5, the UV block cosmetic composition according to an aspect of the present invention had excellent feeling of use.

The UV block cosmetic composition according to an embodiment of the present invention contains UV blocking agents in both the water phase and oil phase, thereby allowing excellent UVA and UVB blocking ability to be achieved.

In addition, the spreadability and powder texture of the cosmetic composition are equal to or higher than those of the hydrophobic powder, and emulsion particles in the composition are homogeneously formed to achieve high emulsification stability, thus leading to excellent feeling of use.

What is claimed is:

1. An ultraviolet (UV) block cosmetic composition in the form of a water-in-oil (W/O) emulsion in which a water phase, as a discontinuous phase, is uniformly distributed in an oil phase, as a continuous phase, or in the form of an oil-in-water (O/W) emulsion in which an oil phase, as a discontinuous phase, is uniformly distributed in a water phase, as a continuous phase, the ultraviolet (UV) block cosmetic composition comprising:
a water phase, an oil phase, and an emulsifier,
wherein the water phase includes a water phase medium and cerium oxide particles, and
the oil phase includes any one or more selected from a group consisting of an oil phase medium, a hydrophobic ultraviolet (UV) blocking agent, and an oil ultraviolet (UV) blocking agent.

2. The UV block cosmetic composition of claim 1, wherein the cerium oxide particles have a secondary particle size of 30 to 200 nm.

3. The UV block cosmetic composition of claim 1, wherein the cerium oxide particles have one or more forms selected from a group consisting of a cubic form, a hexagonal form, a polygonal form, a spherical form, and an aggregated spherical form.

4. The UV block cosmetic composition of claim 1, wherein a content of the cerium oxide particles is 1 to 20 wt % based on the total weight of the UV block cosmetic composition.

5. The UV block cosmetic composition of claim 1, wherein the cerium oxide particles have a powder purity of 98 to 99.9%.

6. The UV block cosmetic composition of claim 1, wherein the cerium oxide particles have a surface charge zeta potential value of 20 to 50 mV.

7. The UV block cosmetic composition of claim 1, wherein the hydrophobic UV blocking agent is one or more selected from a group consisting of zinc oxide particles and titanium dioxide particles.

8. The UV block cosmetic composition of claim 1, wherein a content of the hydrophobic UV blocking agent is 0 to 30 wt % based on the total weight of the UV block cosmetic composition.

9. The UV block cosmetic composition of claim 1, wherein the oil ultraviolet (UV) blocking agent is one or more selected from a group consisting of 4-Methylbenzylidene Camphor, Drometrizole, Drometrizole Siloxane, Digalloyl Trioleate, Disodium Phenyl Dibenzimidazole Tetrasulfonate Acid, Diethylamino Hydroxybenzoyl Hexyl Benzoate, Diethylamino Hydroxybenzoyl Hexyl Benzoate, Diethylhexyl Butamido Triazone, Methylene Bis-Benzotriazolyl Tetramethylbutylphenol, Menthyl Anthranilate, Benzophenone-3, Benzophenone-4, Benzophenone-8, Butyl Methoxydibenzoylmethane, Bis-EthylhexyloxyphenolMethoxyphenyl Triazine, Cinoxate, Ethylhexyl Dimethyl PABA, Ethylhexyl Methoxycinnamate, Ethylhexyl Salicylate, Ethylhexyl Triazone, Octocrylene, Isoamyl p-Methoxycinnamate, Terephthalylidene Dicamphor Sulfonic Acid, Phenylbenzimidazole Sulfonic Acid, Polysilicone-15, and homosalate.

10. The UV block cosmetic composition of claim 1, wherein a content of the oil UV blocking agent is 0 to 25 wt % based on the total weight of the UV block cosmetic composition.

11. The UV block cosmetic composition of claim 1, wherein the water phase medium is purified water or an acidic aqueous solution with a pH of 5 to 7.

12. The UV block cosmetic composition of claim 1, wherein a content of the water phase medium is 10 to 50 wt % based on the total weight of the UV block cosmetic composition.

13. The UV block cosmetic composition of claim 1, wherein the oil phase medium is one or more selected from a group consisting of ethylene glycol, diethylene glycol, propylene glycol, dipropylene glycol, butylene glycol, ester oil, mineral oil, and silicone oil.

14. The UV block cosmetic composition of claim 1, wherein a content of the oil phase medium is 10 to 50 wt % based on the total weight of the UV block cosmetic composition.

15. The UV block cosmetic composition of claim 1, wherein the emulsifier is a non-ionic emulsifier.

16. The UV block cosmetic composition of claim 1, wherein a content of the emulsifier is 1 to 5 wt % based on the total weight of the UV block cosmetic composition.

17. The UV block cosmetic composition of claim 1, further comprising:
a cosmetic formulation composition.

18. A preparation method of an ultraviolet (UV) block cosmetic composition comprising:
dispersing cerium oxide particles in purified water to prepare a supernatant;
adding and stirring the supernatant in a water phase medium to prepare a water phase raw material;
adding and stirring any one or more selected from a group consisting of a hydrophobic ultraviolet (UV) blocking agent and an oil ultraviolet (UV) blocking agent in an oil phase medium to prepare an oil phase raw material; and
stirring an outer phase and injecting an inner phase,
wherein the outer phase and the inner phase are each independently a water phase raw material or an oil phase raw material, and the outer phase and the inner phase are different raw materials from each other.
